(19) United States
(12) Patent Application Publication
(10) Pub. No.: US 2004/0038206 A1
(43) Pub. Date: Feb. 26, 2004

(54) Method for high throughput assay of genetic analysis

(76) Inventors: Jia Zhang, Glendora, CA (US); Kai Li, Glendora, CA (US)

Correspondence Address: Raymond Y. Chan, 1050 Oakdale Lane, Arcadia, CA, 91754, US

(21) Appl. No.: 09/808,659

(22) Filed: Mar. 14, 2001

(51) Int. Cl. C12Q 1/68

(52) U.S. Cl. 435/6

(57) ABSTRACT

Methods for high throughput assay of genetic analysis are provided. Genetic materials of either DNA or RNA are used as the template for primer extension using target specific primers. Following primer extension, the extended products with labeled nucleotides integrated are kept on the solid support and used for visualization and detection. As compared to other methods for genetic assay, this method is quick, reliable, and compatible with analysis of several genetic analysis including polymorphism, gene expression profiling, and sequencing.

### CROSS-REFERENCE TO RELATED APPLICATIONS

This application is a formal application for the DISCLOSURE DOCUMENT NO. 486671: Method for high throughput assay of genetic polymorphism. This application is also cross-referred to a recent filed U.S. patent application Direct measurement of multiple gene expression using antisense single chain array (U.S. patent application Ser. No. 09/758601).

### STATEMENT REGARDING FEDERALLY SPONSORED RESEARCH OR DEVELOPMENT:

Not Applicable.

### REFERENCE TO A MICROFICHE APPENDIX

Not Applicable.

### BACKGROUND OF THE INVENTION

The invention pertains to methods for high throughput assay of genetic analysis by enzymatically extending the target specific primers and then distinguishing the labeled products from primers and unlabeled products. The genetic materials used for polymorphism assay can be either DNA or RNA.

In post-genome era, genetic analysis will focus on genetic polymorphism, gene expression, and whole genome sequencing.

Genetic polymorphism has important scientific impact on studies related to evolution, disease development, and disease prevention. Some diseases, especially genetic diseases, are directly caused by gene defect related to polymorphism. For some other diseases, defects related to genetic polymorphisms are responsible for the higher susceptibility to environmental etiological factors. For the latter situations, it was hypothesized that common disease may have common variant.

The gene sequence information from human genome project makes it possible to identify most of the diseases associated with polymorphisms in the near future. Genetic polymorphism can be single nucleotide polymorphism, deletion or insertion of a piece of genetic sequence, and different number of short repeats. Genetic polymorphism resides in different location such as intron, exon, and promoter region. A different genotype may or may not relate to a different phenotype. If it causes different phenotype, it can be the expression level of a gene product or the different biological activity of a gene product due to the change of protein sequence.

Among different types of polymorphism, single nucleotide polymorphism (SNP) is widely existed. In general, one SNP can be identified in every 1000 nucleotides. The frequency of SNP varies from the types of DNA. For example, the average nucleotide diversity is about 0.11 percent for pseudogenes, 0.10 for introns, and less then one SNP per 1000 nucleotide for coding region.

Many methods have been developed to detect individual polymorphism such as single nucleotide polymorphism (SNP). Among the methods, polymerase chain reaction followed by gel electrophoresis is most frequently used. This method is sensitive and very reliable. Another sensitive method is capillary electrophoresis followed by spectrometry. This method is pretty reliable but it is time consuming and expensive. Recently, a high-density variant detection array using perfect matched oligonucleotides and partially matched oligonucleotides has been made available for polymorphism assay. The advantage of using gene array is quick, efficient, and compatible for high throughput analysis but it is not as sensitive as the two methods mentioned above. More specifically, the high-density variant detection array has a pretty high percentage of false positive (more than 20%) and false negative (nearly 10%) results. In addition, one method called one nucleotide extension has been used in SNP analysis. There is a major drawback for the one nucleotide extension method: it does not work when the sequence between the 3′ end of the detection step primer and the variable nucleotide to be detected is the same.

Different methods have different disadvantage and advantage. Theoretically, the ideal method for polymorphism assay is the one that inherits the advantages of high fidelity of enzymatic reaction and high efficiency of hybridization array. The object of the present invention is to provide methods with the advantage of enzymatic reaction and hybridization array.

The method from this invention can also be applied to expression profiling of multiple gene products and to genetic sequencing of known genes. When less abundant mRNA is assayed, this method can specifically amplify the selected less abundant gene products in a pattern of solid phase primer extension. This solid phase primer extension is more powerful when it is performed after hybridization selection. The post-hybridization primer extension is quite different from conventional RT-PCR. RT-PCR can cause a loss in information of less abundant gene products. However, post-hybridization PCR is specific as the template to be amplified is under double selections: the first selection of hybridization and the second selection of antisense primers.

In brief, the present invention consists of the preparation of target specific primer sets, primer extension using different polymerase upon the template is DNA or RNA, and distinguish the extension products with labeled nucleotide from those of unlabeled products and those of un-extended primers. This method is quick, sensitive, reliable, and compatible with high throughput requirement.

Relevant Literature:

1). Cargill M et al: Characterization of single-nucleotide polymorphisms in coding regions of human genes. Nature Genetics, 22: 231-238, 1999.

2). Halushka M K et al: Patterns of single-nucleotide polymorphisms in candidate genes for blood-pressure homeostasis. Nature Genetics, 22: 239-247, 1999.

3). Lindblad-Toh K et al: Large-scale discovery and genotyping of single-nucleotide polymorphisms in the mouse. Nature Genetics, 24: 381-386, 2000.

4). U.S. Pat. No. 6,013,431

5). U.S. Pat. No. 6,156,501

### SUMMARY OF THE INVENTION

The present invention develops new methods for analysis of one or more genes for providing both qualitative and quantitative information, including genetic polymorphism, gene expression profiling, and sequencing of known genes. Primers targeting specific nucleotides are used in primer extension. Extended products with labeled nucleotides integrated occur only to perfect matched primers. The products from primer extension are then separated using enzymatic or mechanical processes. The primer-extended products with labeled nucleotides precisely document the qualitative and quantitative information of the targeted nucleotide sites after visualization and detection, which then directly reflects the sequence structure of the targeted sites or the expression level of the gene at which the targeted sites resided in.

As to the genetic polymorphism assay, there are two major differences between the methods of this invention and the conventional assay in the prior art. First, the conventional gel electrophoresis used is replaced with enzymatic or mechanical separation of primer extension products, or upgraded using multiple color labeling and size-separation strategies. Second, as compared to the oligonucleotide gene array, the methods in this invention introduces the primer extension reaction to gene array by immobilizing primers on solid support which we named as solid phase primer extension. By these ways, the new methods of this invention significantly increased the sensitivity and reliability in polymorphism assay. This so-called solid phase primer extension is very well compatible to high throughput analysis.

The methods of this invention initiate a new way of assay: analysis of individual differences based on our knowledge of the known sequences from genomic projects. Most conventional assays were focused on detecting unknown sequences of genetic materials. As many genome projects have been done or are nearly done, it is urgently needed in the post genomic era now to develop more efficient methods to screen individual variances. The methods of this invention are developed to meet the requirement of screening known sequences. Briefly, this invention can be used to screen one or more known polymorphism from one sample a time; to screen one or more known polymorphisms from many samples a time; and to detect new polymorphism by sequencing individual genes based on the known sequence information.

### BRIEF DESCRIPTION OF THE SEVERAL VIEWS OF THE DRAWING

FIG. 1 depicts the method using pre-labeled primers for polymorphism assay by enzymatic or mechanic separation.

FIG. 2 depicts the method using unlabeled primers for polymorphism assay by stringency wash.

FIG. 3 representative results using mechanic separation for primer extended products from un-extended primers.

### DETAILED DESCRIPTION OF THE INVENTION

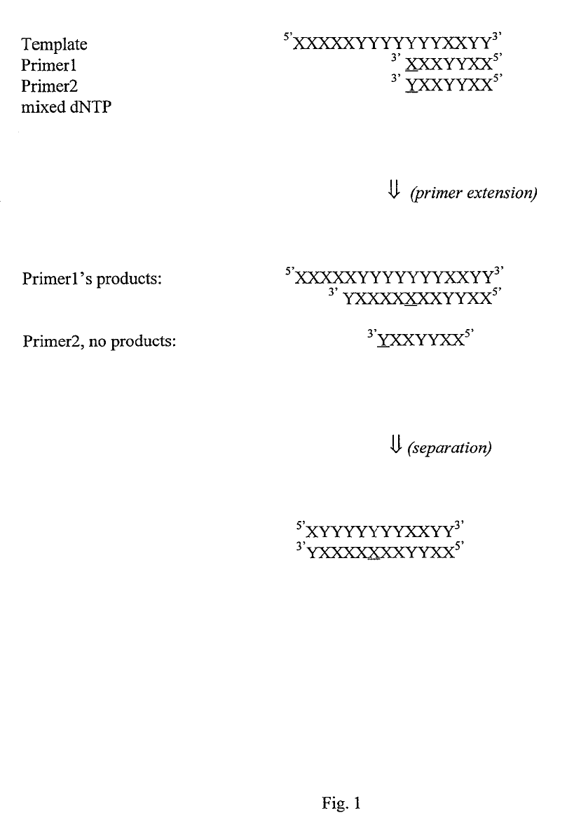

FIG. 1 depicts the method using pre-labeled primers for polymorphism assay. The base on single nucleotide polymorphism site determines whether the base specific primer is extended or not. Non-extended primers are removed with single chain specific exonuclease digestion and/or mechanical removal. Signals from extended products demonstrate the base information of the template used in polymorphism or sequencing analysis.

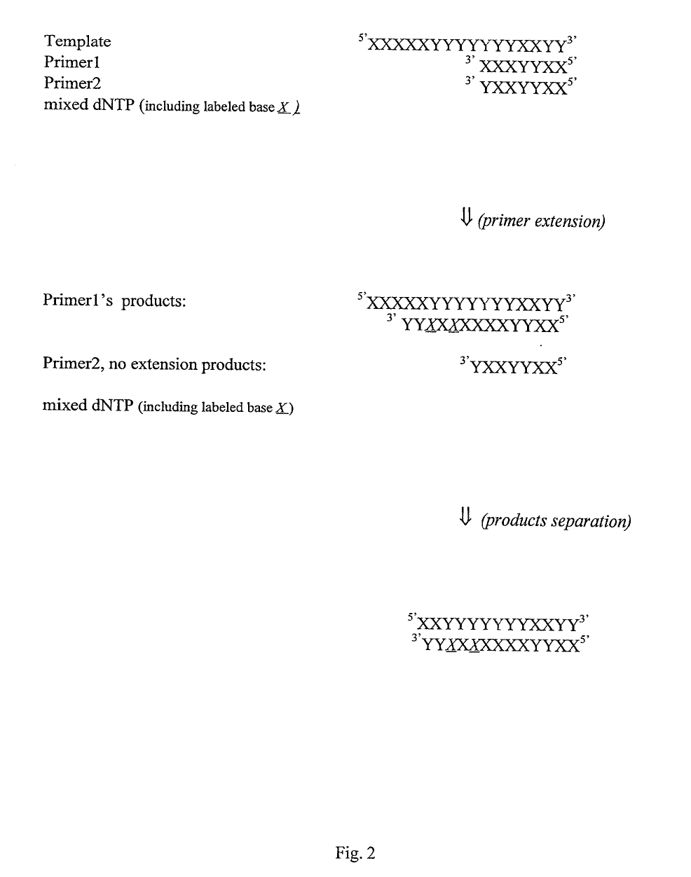

FIG. 2 depicts the method using unlabeled primers for polymorphism assay. Polymerase with no 3′→5′ exonuclease activity is used for primer extension under the condition of mixed dNTPs containing labeled nucleotides. The base difference of a template determines whether the primer extension works or does not. Following primer extension, extensive washes will remove the labeled nucleotide substrate and only the extended products covalently linked to the solid support left, which are then document the base information of the template to be assayed.

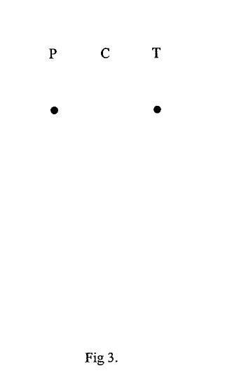

FIG. 3 representative results using mechanic separation for primer extended products from un-extended primers. Human TGF-betal gene has a TC single nucleotide polymorphism, which is associated with some cardiovascular diseases. FIG. 3 illustrated the results using polymorphism base specific primer PCR followed by mechanic separation of the reaction product. The thermocycling procedure used here is same as literature used, consisted of an initial denature at 94° C. for 5 minutes; 35 cycles of denature (94° C. for 30 seconds), annealing (60° C. for 30 seconds), and extension (72° C. for 30 seconds); and a final extension at 72° C. for 5 minutes. The reaction substrate dNTP mix contains dig-labeled UTP. PCR products were then passed through a 30,000 nominal molecular weight cutoff column, and then be visualized. P represents the reaction using all 3 primers together, T represents the reaction using primer 1 for T allele, C represents the reaction using primer 2 for C allele and the primer 3 respectively ((P1, 5′-CTCCGGGCTGCGGCTGCTGCT-3′; P2, 5′-CTCCGGGCTGCGGCTGCTGCC-3′) and 1 antisense primer (P3, 5′-GTTGTGGGTTTCCACCATTAG-3′)). The sample tested is C allele negative.

PCR refers to polymerase chain reaction. A variety of PCR techniques have been developed and primers used in PCR are usually 2 (one pair) or more than 2.

Primer refers to the oligonucleotides complementary specific templates, which is usually in the length of 10 to 50 nucleotides.

Label refers to the incorporation of an easily detectable reagent to nucleic acids, either the immobilized nucleic acids on solid support or the nucleic acids in the solution. In this invention, the label specifically refers to the incorporation of either isotopic or non-isotopic reagents into the single antisense DNA or RNA used for array preparation.

Gene target refers to the gene products to be analyzed, which including DNA and RNA here.

Nucleic acid refers to either DNA (deoxyribonucleotidyl acid) or RNA (ribonucleotidyl acid).

DNA refers to deoxyribonucleotidyl acid. As the genetic material, DNA molecule can be replicated to more molecules and be transcribed to RNA.

cDNA refers to complementary DNA as compared to messenger RNA. cDNA is prepared by in vitro reverse transcription from mRNA.

Genomic DNA refers to DNA in nucleus including both chromosomal and extra chromosomal DNA. Genomic DNA differed from cDNA in its non-coding regions including introns.

RNA refers to ribonucleotidyl acid, including transfer RNA, ribosomal RNA, and messenger RNA. RNA is usually transcribed from DNA but it also can be reverse-transcribed to DNA as seen in retroviruses.

mRNA refers to messenger RNA. Messenger RNA is transcribed from DNA and it is used as a template or model for the synthesis of protein. In experimental condition, mRNA can be used to in vitro reverse transcribe into complementary DNA for the purposes of genetic engineering and for gene expression assays such as used in microarray.

Antisense refers to the genetic sequences complementary to messenger RNA that is defined as sense chain. The rule of the complementary is A to T (U) and C to G.

In vitro transcription refers to the transcription procedure from DNA to RNA carried on in a tube instead of inside a cell. DNA dependent RNA polymerase is required in this process. In vitro transcription is widely used to prepare single chain RNA probe.

Prior to this invention, both enzymatic primer extension and oligonucleotide hybridization have been used in the analysis of polymorphism as mentioned above. These two strategies seem very different and could not been integrated in one method. Primer extension followed by gel electrophoresis is very reliable, which gives a clear answer with yes or no. But this method has several limitations. First, it is not easy to apply to multiple polymorphism sites and in high throughput assay. Second, the editing function from 3′→5′ exonuclease activity of a variety of polymerases may cause false positive results. This false positive potential often requires a further confirmation by sequencing analysis. On the contrary, gene microarray using oligonucleotide hybridization can analyze multiple polymorphisms and is compatible with high throughput assay. However, the latter method is not as sensitive and reliable as the one using primer extension followed by gel electrophoresis. The reason for less sensitive and less reliable of the oligonucleotide hybridization is because its basic principle: identifying polymorphism by comparing the signal/noise ratio based on one base mismatch.

The significant effect of mismatch on hybridization has been very well recognized and widely used in molecular biological studies. The effect of a mismatched base largely depends on where it is located. For the purpose of simple hybridization, one base mismatch at the center of the oligonucleotide has the strongest effect to interfere with the hybridization, but very high variance exists between one oligonucleotide to another as well as between one base to another base. The information obtained from this strategy is higher signal/noise ratio or lower signal/noise ratio. It is impossible to have an answer with yes or no as it could be done with primer extension followed by gel electrophoresis.

When used in a right way, mismatch can provide information more precisely as it works in primer extension followed by gel electrophoresis. For the purpose of primer extension, one or more base mismatch has little effect on primer extension if the mismatched bases are located on the 5′ prime end, as it frequently used in the introducing of restriction enzyme site and mutagenesis. It is recommended that mismatch at any region other than 5′ terminal should be avoided in primer extension. As has been demonstrated in method of assaying polymorphism using primer extension followed by gel electrophoresis, one base mismatch at or near 3′ terminal of a primer almost completely prevented the primer extension to occur, except those products amplified from truncated primers caused by editing. With conventional polymerase chain reaction, primer pairs targeting single nucleotide polymorphism works well. When the primer pair designed in similar size, PCR is run in different tube or well and then separated by gel electrophoresis to determine the homozygo or heterozygo. An alternative for this method is to design the primer pairs in different size so that PCR can run on the same tube and two bands can be distinguished by size after gel electrophoresis.

There is no doubt about the sensitivity and reliability of using primer extension with base-targeted specific primer pairs. The cumbersomeness of gel electrophoresis and one/two reactions per polymorphism site limited its application in high throughput assay of polymorphism. The methods in this invention design specific primer sets to adapt different primer extension reactions. Furthermore, the methods in this invention replace gel electrophoresis by completely distinguishing labeled extended products from those of not labeled extended products and from those of non-extended primers.

As compared to other methods used in assaying genetic polymorphism, the methods of this invention is quick, inexpensive, sensitive, reliable, and compatible to high throughput assay. The methods of this invention can use both DNA and RNA as the template for primer extension. Genome-wide polymorphism can be assayed in hours, which may find use in clinical diagnosis.

It is to be understood that the invention is not limited to the particular embodiments of the invention described below, or to the example of the invention included below.

I. Designing Primer Sets for Primer Extension

The primers used for primer extension are between 18 and 30 nucleotides. gene-specific primers can be unlabeled, 3′ terminal labeled, or randomly labeled to more than one nucleotide. Labeling can be either isotopic or non-isotopic. Two types of primers are designed according to their purposes. The first type of primer is base-specific primer for the assay of known polymorphism. This type of primer set varies from 2 to 4 primers according to the base change of a polymorphism. All nucleotides are similar for primers within a subset except one nucleotide is different near or at the 3′ terminal which could be with either a set of two as A, G; A, C; T, C; and T, G or a set of all the four bases of A, T, C, G.

When a polymorphism is focused on one or more known genes, instead of certain specific sites of the genome as mentioned above, one-base degenerated primer set will be used to cover all known single nucleotide polymorphism sites. The role applied to the last nucleotides at 3′ terminal for the one-base degenerated primer is the same as it is for the base-specific primer. Therefore, the primer set for sequencing a known gene or genes is different from base to base, it could be as less as two specific primers when there is no polymorphism site to be crossed such as in highly reserved regions. On the contrary, in genetic regions where polymorphism exists, the primer set could include 16 or even more degenerated primers for the analysis of one base.

For gene expression profiling, one or more than one gene specific primer are designed for each individual gene.

Whether paired or unpaired primers are used is upon to the type of primer extension. When unidirectional primer extension is performed, only unpaired primers targeting specific bases are required. Enzymes that can be used for unidirectional extension include RNA-dependent DNA polymerase and some DNA-dependent DNA polymerases.

In a specific embodiment where amplification is required, primer pairs have to be provided. In addition to the base-specific primer sets, another type of primer is used to amplify the complementary chain. As the latter primer is only for amplification purpose, it is a perfect match primer. In general, one such kind of perfect primer can serve for 50 to 500 sets of base-specific primers. The number of the perfect matched primer needed is upon to the length of the gene to be assayed as well as the extension duration to be set. It is always good to keep the extension product shorter than 500 base pairs, and preferably shorter than 200 base pairs. Primer extension with amplification can also be performed using primer pairs of base specific primer and universal primer. Theoretically, universal primer works for all types of primer extension. But the combination of universal primer and unlabeled base-specific primer will cause some background and therefore decrease the signal/noise ratio. It is simple and powerful to only use universal primer when labeled base-specific primers are used.

II. Primer Extension

The template for primer extension can be any kind of nucleic acid, either directly isolated from biological sample such as genomic DNA and RNA, or in vitro transcribed cDNA. Different type of template requires different polymerase: RNA dependent DNA polymerase is used for RNA template and DNA dependent DNA polymerase is used for DNA template including both genomic DNA and complementary DNA. When genomic DNA is used as a template, a brief sonification or partial digestion with EcoR I of genomic DNA will increase the efficiency of primer extension.

There are two types of primer extension reactions employed in polymorphism assay: unidirectional primer extension and polymerase chain reaction. Primers can be pre-labeled or non-labeled. Unidirectional primer extension only needs base-specific primers. Polymerase chain reaction requires base-specific primers and another primer for the amplification of the complementary chain of the template. Temperatures and other specific settings for using different polymerases are well known in the arts.

Primers can be covalently cross-linked to the surface of the solid support or be dissolved in the reaction buffer during the primer extension. A variety of cross-linking methods are well known in the art including baking, UV cross-linking, and chemical reactions through specific reagents. Non-labeled primers and 3′ terminal labeled primers can be used in either way, cross-linked on the surface of the solid support or dissolved in the reaction buffer. But randomly labeled primers with more than one nucleotide labeled cannot be cross-linked on the solid support as this may prevent from the completely separation of wanted signal in extended products from unwanted signal of the labeled primers.

The solid phase primer extension has lower efficiency when target specific primers are immobilized as compared to regular primer extension completely performed in liquid phase. This feature of solid phase primer extension can be partially overcome by increasing the amount of template when non-labeled primers are immobilized. For 3′ terminal labeled primers, a mixture of all non-labeled perfect matched primers from the different target specific primer sets is dissolved in the liquid phase. Preferably, these perfect matched primers are 1 to 5 nucleotides shorter at their 3′ as compared to those to be immobilized. The amount of these primers is between 1/100 and 1/1000 of the immobilized ones. This protocol is especially helpful when the template is rare in the amount when cDNA is used as a template. Actually, this step integrates two primer extension reactions into one system: amplification of template of interested and polymorphism analysis. Having two types of primer extension in one system provides many advantages. It shortens the analysis duration, decreases expenses, and decreases contamination chances. Increasing cycling number is another option to increase the sensitivity of polymorphism analysis. In most situations, 30 to 50 cycles are used for solid phase primer extension.

Without optimization, it is hard to have one primer extension reaction in liquid phase and simultaneously have another primer extension at solid phase. Although the primer extension in solid phase has exactly the same product as the one in liquid phase, their templates are different. The sample to be analyzed is used as the template for liquid phase primer extension initially. The template for solid phase primer extension is mainly the product from the liquid phase primer extension. By this reason, these two reactions are defined as cascade primer extension.

One key parameter for primer extension is annealing temperature, which is based on the calculation as the following: Tm=4×(G+C)+2×(A+T). For example, if a primer has 10 nucleotides of G and C, and 10 nucleotides of A and T, the annealing temperature would be around 60° C. Practically, the annealing temperature can be adjusted between 55 and 65° C. in this example according to the type of polymerase to be used.

This calculation for annealing temperature mentioned above is solely for liquid phase primer extension, where both template and primers are in free forms. The hydrogen bonds formed between primer and template is strong enough to protect the small primer from dissociation: namely, the force from primer's random movement is weaker than the whole force of the hydrogen bonds between primer and template. Therefore, in liquid phase primer extension, the annealed primer and the template behave as one molecule in their random movement at optimal annealing temperature.

However, the annealing temperature calculated for liquid phase primer extension does not work for solid phase primer extension. Since the target specific primer is immobilized on a solid support, it cannot behave as a single molecule with a large size template in random movement. The force from the hydrogen bonds between the target specific primer and the template especially genomic DNA is not enough to restrain a large size template from dissociated from a primer. In order to approach the annealing temperature used for liquid phase primer extension, increasing the length of primers immobilized on a solid support and decreasing the size of template to be used are the solution. In most cases, genetic materials to be analyzed for polymorphism are very large. Even after partial digestion, genomic DNA is not compatible for solid phase primer extension as no optimal annealing temperature could be found. By using cascade primer extension strategy, the liquid primer extension provides short template for solid phase primer extension. The latter is the real one for polymorphism analysis.

In a specific embodiment when base-specific primers are dissolved in reaction buffer, primer extension is performed in physically separated microwells. Each microwell can contain one or more base-specific primers based on what kind of labeling is used and how many types of different labeling are employed. The specific setting of conditions for primer extension is variable upon to the templates. A 5 to 10 minutes denature is needed when genomic DNA is used, whereas, initial long denature step is usually not required when cDNA or RNA is used as the template. The cycling number is set between 20 and 40. Since cross-linkage partially decreased the hybridization ability of the primers, primers dissolved in reaction buffer increase both the specificity and efficiency of the reaction.

In general, polymerase chain reaction with amplification provides higher efficiency as compared to unidirectional primer extension. Commercial available thermocycler can meet the requirement for polymerase chain reaction in the formats of either microwell plates or microscope slides (MJ Research, ALP-1238 for 384-well plate and ALD-0211 for 32-microslides).

Two types of primer extensions can be used for gene expression profiling. The first type uses in vitro transcribed cDNA as template. This type gene profiling is convenient but it does not increase assay specificity as compared to regular RT-PCR. In this case, reverse-transcription is first performed before primer extension. The cycles of primer extension needs to be controlled based on the redundancies of the genes to be analyzed. Using more than one arrays run at different cycles will provide more precise quantitative information for wide range of genes. For gene expression profiling, primer pairs should targeting to one or more specific sites of a given gene: one pair targeting to region near 5′ end and another pair targeting to region near 3′ terminal of the cDNA.

The more sophisticated gene expression profiling is post-hybridization primer extension, which uses antisense DNA chain as template. During the procedure of array preparation, selected probes of modified oligonucleotides with nuclease resistance are co-plotted with single chain antisense. The latter is subjected to single chain nuclease digestion after hybridization. The survived antisense single chain is then amplified. This type of post-hybridization primer extension combined the amplification power of PCR and the high specificity of hybridization. Conventional array has PCR first and then followed by hybridization, which usually amplify the abundant gene products. The method of this invention reversed the conventional procedures: hybridization first and then specific amplification of selected less abundant gene products.

III. Removal of Unwanted Signal

High background noise is always the problem to be solved in the application of gene array. Some common noise such as non-specific binding between labeled gene target/probe and the solid support, non-specific duplex formation between gene target and probe are completely eliminated by the novel strategy utilized in the methods of this invention.

The only unwanted signal to be removed before visualization and detection is the labeled nucleotides not integrated into the primer extended products. This type of unwanted signal presents three different forms depending on what kinds of primer sets are used.

In situations when unlabeled primer sets are used for primer extension, the unwanted signal is the labeled nucleotides, the substrate of primer extension reaction. It is appreciated that single nucleotides can be simply washed out from the extended products that are covalently cross-linked to the solid support via the primers. Using high stringency wash buffer such as 0.5×SSC, 3×5 minutes is usually enough to remove the labeled single nucleotides. Unlabeled primers have unique application in quantitative analysis on gene expression profiling. When modified oligonucleotide with nuclease resistance are plotted together with prelabeled single chain antisense (detailed in U.S. patent application Direct measurement of multiple gene expression using antisense single chain array (USPO 09/758601), nuclease protected single chain DNA can be then amplified by solid phase PCR after adding a mixture of selected antisense primers. This kind of post-hybridization PCR will maximize the signals of less abundant mRNA. Since hybridization process is highly specific, the post-hybridization PCR will only increase signal with minimal increase on noise, thereby increase the sensitivity. Another advantage of the post-hybridization PCR is that the antisense primers can be selected in each run with one type of labeling. Sequential run with selected antisense primers, either labeled or unlabeled, can be used to specifically amplify the less abundant mRNA. For post-hybridization primer extension, a renature with gradually cooling down is needed before further wash. Any unwanted signal can be easily washed out using different stringency buffer after solid phase PCR is performed.

As mentioned in the section of primer extension, the 3′ terminal labeled primers can be used in two different ways: cross-linked on solid support and dissolved in the primer extension buffer. After primer extension, application of single chain specific enzyme digestion will remove the labeled 3′ terminal nucleotide. S1 nuclease at the concentration of 100 units/ml for 5 to 10 minutes at the temperature of 30 to 35 degree centigrade is sufficient to remove the labeled 3′ terminal nucleotide. Following S1 nuclease digestion, the digested nucleotides are removed with 3×5 minutes wash using 0.5×SSC when the primer sets are cross-linked on the solid support. In a specific embodiment when the 3′ terminal labeled primers are dissolved in primer extension buffer, mechanic separation of the extended products from the primers by size, through columns or membranes, will be followed in order to remove the labeled primers. In this case, S1 digestion can be done before mechanical separation but this step is not required.

Two further explanations for S1 nuclease digestion are needed. When applying S1 nuclease digestion, primer extended products has to be gradually cooled down to room temperature for 10 to 30 minutes. This step is to allow the duplex formation between the two strands of nucleic acids. This step of S1 nuclease digestion can be avoided when using polymerase with strong 3′→5′ exonuclease activity. The 3′ terminal labeled nucleotide of the mismatched primer is removed during primer extension. In order to reach the zero background standard, including S1 nuclease digestion is always suggested.

Mechanical separation will be employed to remove the primers with several nucleotides labeled. After primer extension, a quick spin or vacuum will be applied to gene arrays such as in the format of microwell plates that are specialized for certain molecular size cutoff column or membrane integrated. Extensive wash is required to completely remove the labeled primer. TE buffer or 0.1×SSC 5 to 10 times wash is usually enough.

Without updating the technique, conventional gel electrophoresis is not able to separate results from large number of SNP such as more than 1000 or more than 10,000 SNP. This invention uses different color labeling combined with size-specified PCR products, which maximized the differentiation capability of gel electrophoresis. For example, each color of florescent labels 50 to 200 pairs of primers that designed for extending different size-specific products, a single 96-well PCR can detect up to 9,600 SNP sites for a single color. Given 4 or 10 different labeling methods are used, the power of detecting SNP will extend to 4×9600 or 10×9600.

In circumstance when florescent scanner is not available, radio-labeling combined with 96-well PCR reaction still works fine. The power is similar to the single color florescent labeling.

IV. Visualization and Detection

Additional processes are only required for protocols using non-isotopic and non-fluorescent labeling. Labeled signal integrated into primer extended products can be detected using specific methods based on the labeling techniques. Examples of detection methods include those for non-isotopic labeled probes such as fluorescence measurement, light emission measurement, and calorimetric measurement; and those for isotopic labeled probes such as autoradiography and scintillation counting. These detection methods are well known to those of skill in the art familiar to the particular signal producing system employed.

What is claimed is:

1). A method for high throughput assay of genetic analysis, comprising the steps of:
(a) preparing target specific primer sets;
(b) performing primer extension;
(c) distinguishing the extended products containing the labeled nucleotide from the extended products without labeled nucleotide and from non-extended primers; and
(d) detect the labeled product from primer extension left on the solid support.

2). The method according to claim 1, wherein said primer sets are paired primers.

3). The method according to claim 1, wherein said primer sets are unpaired primers.

4). The method according to claim 1, wherein said primer sets are unlabeled.

5). The method according to claim 1, wherein said primer is unmodified oligonucleotides.

6). The method according to claim 1, wherein said primer is modified oligonucleotides.

7). The method according to claim 1, wherein said primer is labeled at the 3′ terminals.

8). The method according to claim 1, wherein said primer is labeled at more than one nucleotides.

9). The method according to claim 1, wherein said target specific primers sets are primers consisting of different subsets of primers with similar nucleotide sequences except at least having one mismatched nucleotide A, T, C, G respectively.

10). The method according to claim 9, wherein said target specific primer sets are immobilized on a solid support before primer extension.

11). The method according to claim 9, wherein said target specific primer sets are added into a liquid phase for primer extension.

12). The method according to claims 10, wherein said primer extension is solid phase primer extension.

13). The method according to claims 10, wherein said primer extension is cascade primer extension.

14). The method according to claims 10, wherein said primer extension is post-hybridization primer extension.

15). The method according to claim 1, wherein said primer extension is performed at 37 degree centigrade.

16). The method according to claim 1, wherein said primer extension is performed at temperature higher than 37 degree centigrade.

17). The method according to claim 1, wherein said primer extension is performed with DNA polymerase including DNA dependent DNA polymerase and RNA dependent DNA polymerase.

18). The method according to claim 1, wherein said primer extension is performed using unlabeled substrates of dNTPs.

19). The method according to claim 1, wherein said primer extension is performed using unlabeled dNTPs mixed with labeled nucleotides.

20). The method according to claim 1, wherein said extended product containing labeled nucleotide is separated from the un-extended primer using enzymatic treatment.

21). The method according to claim 1, wherein said extended product containing labeled nucleotide is mechanically separated from the un-extended primer.

22). The method according to claim 1, wherein said extended product containing labeled nucleotide is distinguished from the extended product without labeled nucleotide by visualization and detection.
